(19) United States
(12) Patent Application Publication
(10) Pub. No.: US 2022/0168233 A1
(43) Pub. Date: Jun. 2, 2022

(54) HYDROGEL/NANOPARTICLE COMPLEX WITH TEMPERATURE SOL-GEL TRANSITION FOR SUSTAINED DRUG RELEASE

(71) Applicant: Tgel Bio Co., Ltd., Seoul (KR)

(72) Inventors: Keun Sang OH, Daejeon (KR); Han Weon CHO, Seou1 (KR); Chang Soon HWANG, Incheon (KR); Sun Jong KIM, Seoul (KR); Hye Sook CHUNG, Seoul (KR); Eun Sook KIM, Daejeon (KR)

(21) Appl. No.: 17/107,544

(22) Filed: Nov. 30, 2020

(51) Int. Cl. A61K 9/51; A61K 9/06

(52) U.S. Cl. CPC A61K 9/5192; A61K 9/06; A61K 9/5146; A61K 9/5161; A61K 9/5123

(57) ABSTRACT

The present disclosure relates to a temperature-sensitive nanoparticle/hydrogel complex, and more particularly, the temperature-sensitive nanoparticle/hydrogel complex for sustained-release drug release that can delay the drug release rate at the administration site, thereby maximizing the drug treatment efficacy at the local site by the interaction between the drug and the nanoparticles as well as the interaction between the drug and the hydrogel when mixing a drug-free nanoparticle/hydrogel complex and a predetermined drug.

### TECHNICAL FIELD

The present disclosure relates to a temperature-sensitive nanoparticle/hydrogel complex, and more particularly, the temperature-sensitive nanoparticle/hydrogel complex for sustained-release drug release that can delay the drug release rate at the administration site, thereby maximizing the drug treatment efficacy at the local site by the interaction between the drug and the nanoparticles as well as the interaction between the drug and the hydrogel when mixing a drug-free nanoparticle/hydrogel complex and a predetermined drug.

### BACKGROUND

Hydrogel can absorb a large amount of water by swelling with water in an aqueous solution, and has a cross-linked network structure in three-dimensions. The network structure in three dimensions is formed through physical bonding such as hydrogen bonding and a van der Waals bonding or chemical bonding such as Ion bonding and covalent bonding. Hydrogel has high biosynthesis through high moisture content as well as physical/chemical similarity of extracellular matrix. With these characteristics, hydrogel has received considerable attention in medical and pharmacological applications.

In particular, the porous structure of hydrogel makes it easy to load a drug onto the gel, and has an advantage that the drug is slowly released depending on diffusion coefficient of the drug. Thus, being applied in the field of drug delivery, hydrogel makes surrounding tissues continuously maintain a specific drug concentration over a long period of time.

However, despite of these advantages of hydrogel, there are several disadvantages which limit practical applications. First, low tensile strength of hydrogel limits application in areas where it has to endure weight, thereby disappearing from the target point through rapid dissolution. Further, it is difficult to carry a hydrophobic drug due to the high moisture content and porosity of hydrogel, in addition to the problem of relatively quick release. Further, most hydrogel is difficult to administer by injection, thereby requiring a surgical operation to introduce it into the body. With these disadvantages, hydrogel is practically limited in clinical use.

The aim of research to solve these problems is to improve the interaction between the drug and the hydrogel, and to slow the spread of a drug loaded in the hydrogel. Several methods have been researched to control the drug release rate, such as modifying the surface of the hydrogel or the microstructure of the entire gel.

This relatively dense hydrogel matrix is formulated to provide properties that can be controlled and has excellent mechanical properties, for improved drug loading efficiency compared to conventional hydrogel.

The drug carrier as described above depends on the polymer concentration of the carrier so that after it is applied to the body it is easily soluble in water, thus causing rapid drug release due to its imperfection, especially when water-soluble drugs or protein drugs and antibodies are being delivered, so it is difficult to implement the desired medicinal effect due to high solubility in aqueous solution. As a way to address this issue, repeated drug administration is required, and in the case of expensive protein drugs or antibodies, the cost of treatment will go up. In order to overcome this issue, various nanotechnology products containing drugs in a temperature-sensitive polymer were mixed and used, however they did not seek to increase efficacy due to the absence of polymers and nanoparticles.

Thus, it is required to do research for a method for a drug carrier with excellent biocompatibility and slow release time and speed of the drug.

According to these needs, in the developed conventional technology, the preparation involves including the drug in the process of making nanoparticles. One such technology is disclosed in Korean patent publication 10-2017-0110204. As disclosed in these documents, the drug to be delivered is loaded on nanoparticles in advance, to form medicine, after which the medicine is stored, and used as required. However, these methods have problems in that the various drugs have to be manufactured separately for each of the necessary drugs and the drugs are difficult to store.

Thus, it is the time to research methods of mixing between the prepared drug carrier (without drugs) and medication needed for patients as required.

### SUMMARY

The present disclosure is to provide a temperature phase transition nanoparticle/hydrogel for sustained drug release drug and a method for preparing the same, which may induce sustained drug release through simple mixing with a predetermined drug.

Further, the present disclosure is to provide a temperature phase transition nanoparticle/hydrogel for sustained drug release drug and a method for preparing the same, which may control drug release by formation of a solid gel through a sol-gel phase transition phenomenon at a temperature similar to body temperature and by the interaction between a drug and hyaluronic acid and interaction of a drug and nanoparticles.

An aspect of the present disclosure provides a method of preparing a temperature phase transition nanoparticle/hydrogel.

In an example of the present disclosure, the method comprises the steps of: forming a first mixture by mixing an aqueous lecithin solution and polysorbate 80; forming a second mixture including nanoparticles by irradiating ultrasonic waves to the first mixture; forming a third mixture by mixing the second mixture and an aqueous poloxamer solution; forming a fourth mixture by mixing the third mixture and an aqueous hyaluronic acid solution; and forming a nanoparticle/hydrogel complex by freeze-drying the fourth mixture.

In an example of the present disclosure, the mixing ratio (weight ratio) of the lecithin and polysorbate 80 is 1:0 to 2 in the step of forming the first mixture.

According to the exemplary embodiments of the present disclosure, the average particle diameter of the nanoparticles is 40 nm to 250 nm in the step of forming the second mixture.

In an example of the present disclosure, the poloxamer is a poloxamer 407.

In an example of the present disclosure, the mixing ratio (weight ratio) of lecithin and polysorbate 80, and poloxamer in the second mixture is 1:2 to 20 in the step of forming the third mixture.

In an example of the present disclosure, the aqueous hyaluronic acid solution includes hyaluronic acid.

Another aspect of the present disclosure provides a temperature phase transition nanoparticles/hydrogel complex.

In an example of the present disclosure, the complex comprises: an irregular mesh-shaped hyaluronic acid network; and nanoparticles including lecithin, polysorbate 80, and poloxamer 407 located between the network, in which the mixing ratio (weight ratio) of the hyaluronic acid and poloxamer 407 is 1:20 to 200.

In an example of the present disclosure, a weight ratio of the lecithin, polysorbate 80:poloxamer 407:hyaluronic acid is 1:0.1 to 2:2 to 20:0.01 to 1.

In an example of the present disclosure, the nanoparticle/hydrogel complex is a sol state at room temperature, but a gel state at 31° C. or higher.

In an example of the present disclosure, the nanoparticle is a lipid-based nanoparticle.

According to an exemplary embodiment of the present disclosure, it is capable of providing a temperature-phase transition nanoparticle/hydrogel complex that maintains a sol state outside the body and maintains a gel state in the body when injected.

According to an exemplary embodiment of the present disclosure, it is capable of providing a temperature-phase transition nanoparticle/hydrogel complex that may locally inject the same by applying a nanoparticle size.

According to an exemplary embodiment of the present disclosure, it is capable of providing a temperature-phase transition nanoparticle/hydrogel complex in which, when mixed with a predetermined drug and administered together into the body, the effective mixed drug interacts with the nanoparticles, thereby continuously releasing the drug.

According to an exemplary embodiment of the present disclosure, it is capable of providing a temperature-phase transition nanoparticle/hydrogel complex that can be decomposed in the body or quickly released outside the body by applying lipid-based nanoparticles derived from natural substances.

According to an exemplary embodiment of the present disclosure, it is capable of providing a temperature-phase transition nanoparticle/hydrogel complex that has excellent sustained-release characteristics by simply mixing with a drug.

According to an exemplary embodiment of the present disclosure, it is capable of providing a temperature-phase transition nanoparticle/hydrogel complex that has excellent sustained-release characteristics by simply mixing with a water-soluble drug by introducing nanoparticles capable of interacting with a drug.

According to an exemplary embodiment of the present disclosure, it is capable of providing a temperature-phase transition nanoparticle/hydrogel complex in which the complex is composed of a block copolymer polymer, lecithin, and hyaluronic acid forms the temperature phase transition nanoparticles through the interaction between the contained polymer and lecithin so as to promote sustained-release drug release through polar interactions with drugs and to enhance the physical properties of hydrogels and promote sustained-release drug release through interactions between hyaluronic acid and nanoparticles having a long chain structure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a flow chart for a method of preparing a nanoparticle/hydrogel complex according to an embodiment of the present disclosure;

FIG. 2 is a schematic diagram of a method of preparing a nanoparticle/hydrogel complex for sustained-release drug delivery according to an embodiment of the present disclosure;

FIG. 3 is a schematic diagram illustrating a sol-gel phase transition phenomenon according to an increase in temperature of a nanoparticle/hydrogel complex for sustained-release drug delivery according to an embodiment of the present disclosure;

FIG. 4 is a view showing the results of a scanning electron microscope according to the formation of a temperature phase transition nanoparticle/hydrogel complex for sustained-release drug delivery;

FIG. 5 is a graph showing the results of changes in mechanical properties according to the temperature change of a temperature phase transition nanoparticle/hydrogel complex for sustained-release drug delivery (Example 1);

FIG. 6 is a graph showing the results of the change in mechanical properties according to the temperature change of the hydrogel (Comparative Example 1);

FIG. 7 is a graph showing a comparison result of changes in mechanical properties of Example 1 and Comparative Example 1;

FIG. 8 is a graph showing the drug release behavior of Example 2 and Comparative Example 2; and

FIG. 9 is a graph showing drug release behavior according to changes in the content of lecithin in Examples 3 and 4 and Comparative Examples 3 and 4.

### DETAILED DESCRIPTION

The terms used in the present disclosure are only used to describe specific embodiments and are not intended to limit the present disclosure. Singular expressions include plural expressions unless the context clearly indicates otherwise. In the present disclosure, terms such as “include” or “have” are intended to designate that features, elements, etc. described in the specification, but does not mean that one or more other features or elements may not exist or be added.

In addition, terms such as “first” and “second” used herein are merely used for the purpose of distinguishing parts to be described later from each other, and do not limit the parts to be described later.

Unless otherwise defined, all terms, including technical or scientific terms, used herein have the same meaning as commonly understood by one of ordinary skill in the art to which the present disclosure belongs. Terms as defined in a commonly used dictionary should be interpreted as having a meaning consistent with the meaning in the context of the related technology, and should not be interpreted as an ideal or excessively formal meaning unless explicitly defined in the present disclosure.

In the present disclosure, the term “nano” may mean a size in a nanometer (nm) unit. For example, it may mean a size of 1 nm to 1,000 nm, but is not limited thereto. In addition, in the present specification, the term “nanoparticle” may mean a particle having an average particle diameter in a nanometer (nm) unit, and for example, it may mean a particle having an average particle diameter of 1 nm to 1,000 nm, but it is not limited thereto.

Hereinafter, a method of preparing a nanoparticle/hydrogel complex for sustained-release drug delivery according to an embodiment of the present disclosure will be described in detail with reference to the accompanying drawings. However, the accompanying drawings are illustrative, and the scope of a method of manufacturing a nanoparticle/hydrogel complex for sustained-release drug delivery according to an embodiment of the present disclosure is not limited by the accompanying drawings.

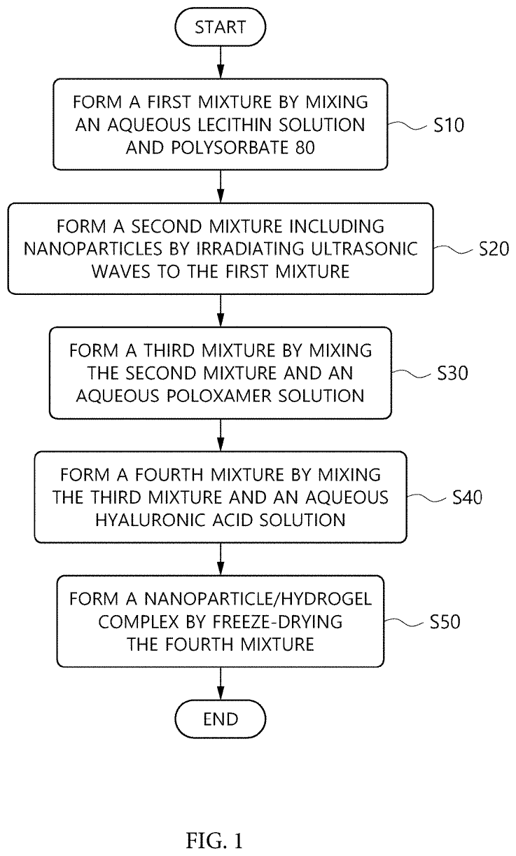

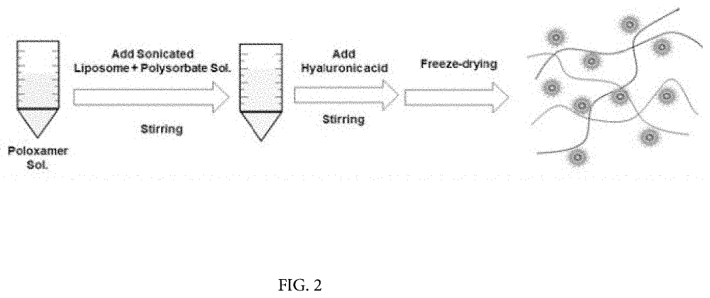

FIG. 1 is a flow chart for a method of preparing a nanoparticle/hydrogel complex according to an embodiment of the present disclosure. FIG. 2 is a schematic diagram of a method of preparing a nanoparticle/hydrogel complex for sustained-release drug delivery according to an embodiment of the present disclosure.

As shown in FIGS. 1 and 2, the method of preparing a nanoparticle/hydrogel complex comprises the steps of: (S10) forming a first mixture by mixing an aqueous lecithin solution and polysorbate 80; (S20) forming a second mixture including nanoparticles by irradiating ultrasonic waves to the first mixture; (S30) forming a third mixture by mixing the second mixture and an aqueous poloxamer solution; (S40) forming a fourth mixture by mixing the third mixture and an aqueous hyaluronic acid solution; and (S50) forming a nanoparticle/hydrogel complex by freeze-drying the fourth mixture.

Hereinafter, a method of preparing a nanoparticle/hydrogel complex according to an embodiment of the present disclosure will be described in detail step by step.

S10: an aqueous lecithin solution and polysorbate 80 are mixed to form the first mixture (the mixture of polysorbate 80 and lecithin).

Lecithin is a term referring to a group of yellowish-brown fatty substances occurring in animal and plant tissues, consisting of phosphoric acid, choline, fatty acids, glycerol, glycolipids, triglycerides, and phospholipids. The phospholipid has a structure having a hydrophilic head and a hydrophobic tail and forms spherical liposomes through hydrophobic bonds between the hydrophobic tails in an aqueous solution. The hydrophilic head has both positive and negative polarities, but overall negative polarity. The phospholipid may have a structure represented by the following chemical formula 1.

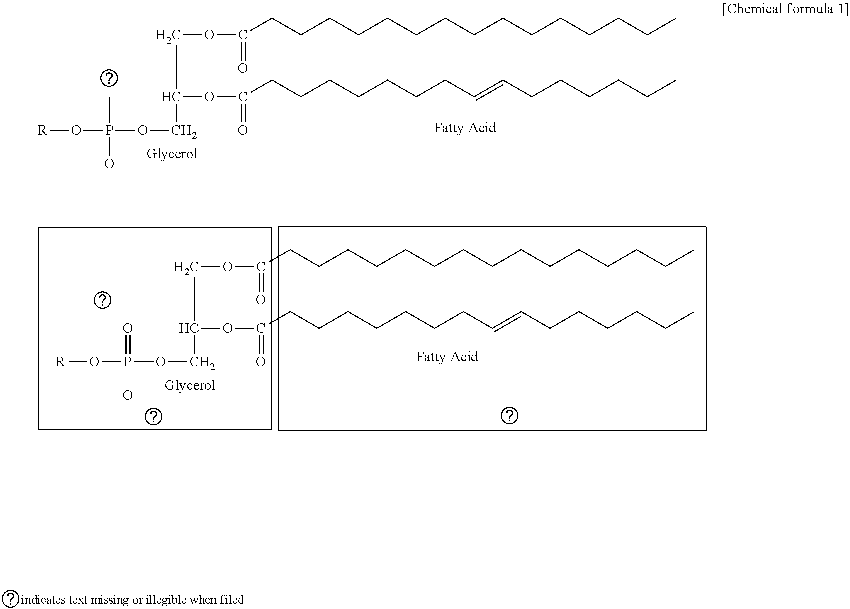

Further, when an aqueous solution containing lecithin is delivered to the body through the joint cavity, it acts as a boundary lubricant by the mechanism of hydration-lubrication, reducing the pressure generated in the cartilage tissue, thereby helping cartilage tissue regeneration. Since the temperature phase transition nanoparticle/hydrogel complex contains lecithin as a constituent component, it may also help regenerate cartilage.

The solvent of the aqueous lecithin solution is not particularly limited, but any solvent suitable for dissolving lecithin may be applied. As an example of such a solvent, distilled water may be used.

Here, the concentration of the aqueous lecithin solution is not particularly limited, and as described later, the ratio of the final content of the lecithin and the content of the poloxamer is important. Meanwhile, the concentration of the aqueous lecithin solution may be 5% to 95%, 10% to 90%, 15% to 85%, 20% to 80% and 25% to 75% as an example, and as described above, the concentration of the aqueous solution may be controlled and used according to the content as intended by the present disclosure.

Polysorbate 80 is an additive used to uniformly disperse liquids or solids, which are not well mixed with each other, in a liquid. By mixing polysorbate and an aqueous lecithin solution, the size of liposomes contained in the aqueous lecithin solution can be made smaller.

Here, the mixing ratio (weight ratio) of lecithin and polysorbate 80 is preferably 1:0 to 2 (excess of 0). The reason for adding polysorbate 80 is that, as described later, when polysorbate 80 is applied to lecithin, and then ultrasonic waves are applied thereto, the nanoparticle size is reduced (for example, 100 nanometers to 30 nanometers), securing more uniform size of the same. When the size of the nanoparticles decreases, a nanoparticle/hydrogel complex that allows the drug to be released more slowly can be expected as the polar interaction with the drug increases. On the other hand, when the mixing ratio of the polysorbate 80 exceeds 2, an increase in the intended effect of the present disclosure may be reduced, and the harmfulness to the human body may be relatively high.

S20: ultrasonic waves are irradiated to the first mixture to form a second mixture including nanoparticles (a mixture of nano-sized polysorbate 80 and lecithin).

The method of irradiating ultrasonic waves is not particularly limited, and a known method of irradiating ultrasonic waves may be applied. Preferably, any method may be applied unless the method deviates from the scope of the intended invention. Most preferably, a probe-type ultrasonic irradiation method would be used.

By irradiating ultrasonic waves, the first mixture is crushed to form nanoparticles having a smaller size, wherein the average particle diameter of the nanoparticles may preferably be 40 nm to 250 nm, 50 nm to 240 nm, 60 nm to 230 nm, 70 nm to 220 nm, 80 nm to 210 nm, 90 nm to 200 nm, 100 nm to 190 nm, 110 nm to 180 nm, or 120 nm to 170 nm.

After nano-sized particles are formed and finally freeze-dried as described below, when mixed with a predetermined drug, the drug can be better loaded, and rapid drug release can be overcome by utilizing the polarity interaction through the aforementioned nanoparticles. Thus, it is possible to provide a nanoparticle/hydrogel complex developed from conventional hydrogel.

S30: the second mixture and an aqueous poloxamer solution are mixed to form a third mixture (a mixture of lecithin and poloxamer).

The term “temperature-sensitive” as used herein refers to a phenomenon of sol-gel phase transition in which a liquid sol is changed to a solid gel under a specific temperature depending on the concentration of the aqueous polymer solution, which is due to the increase in the attraction between the nanoparticles as the temperature of the nanoparticles containing the poloxamer with temperature sensitivity increases.

Further, in order to induce polar interactions with drugs along with temperature-sensitive polymers exhibiting sol-gel phase transition, lecithin which has both negative and positive polarities is used with nanoparticles, and when mixed with drugs through the polarity of liposomes, sustained release can be induced through interaction with drugs.

The above-described temperature-sensitive polymer refers to a polymer capable of exhibiting a sol-gel phase transition phenomenon, and specifically, may be a poloxamer or a pluronic.

The poloxamer or pluronic may be a block copolymer polymer represented by the following formula.

HO—(C₂H₄O)a-(C₃H₆O)b-(C₂H₄O)a-H [Chemical formula]

It has the official molecular weight of 12.6 kDa, and has a chain structure in which a polyethylene oxide (PEO) block (a) 101 and a polypropylene oxide (PPO) block (b) 56 are repeatedly connected. The poloxamer, block copolymer polymer, has a structure of polyethylene oxide (PEO)-polypropylene oxide (PPO)-polyethylene oxide represented by the above formula in which the polypropylene oxide in the middle is a hydrophobic portion, and polyethylene oxides on both sides are hydrophilic portions.

Here, the poloxamer may be poloxamer 407 or pluronic F-127.

Therefore, the nanoparticles used in the present disclosure are formed using block copolymer polymers having both hydrophobicity and hydrophilicity and liposomes. Block copolymer polymers having both hydrophobicity and hydrophilicity are poloxamers or pluronics, which possess both hydrophilic and hydrophobic portions. In the aqueous solution, the affinity of polyethylene oxide, which is a hydrophilic portion, with water molecules, and the repulsion of polypropylene oxide, which is a hydrophobic portion, with water molecules, form a structure called a micelle, which has a hydrophobic portion inside and a hydrophilic portion outside.

Since both poloxamer and lecithin have hydrophobic and hydrophilic portions, the polypropylene oxide of poloxamer and the tail of phospholipids are bonded to form nanoparticles through the hydrophobic bond between the hydrophobic portions and the repulsion force with the water molecule of the hydrophobic portions.

Since the formed nanoparticles contain poloxamer, allowing the sol-gel phase transition phenomenon, which is a characteristic of poloxamer, a temperature-sensitive polymer. Thus, the attraction between the nanoparticles is strengthened at a temperature similar to body temperature so that the liquid sol becomes a solid phase and also its physical properties are strengthened.

Here, the concentration of the poloxamer aqueous solution is not particularly limited, and as described below, the final ratio of the content of lecithin and the content of poloxamer is critical. Meanwhile, the concentration of the poloxamer aqueous solution may be 5% to 95%, 10% to 90%, 15% to 85%, 20% to 80%, and 25 to 75% as an example, and as described above, the concentration of the aqueous solution can be controlled and used so as to match the content as intended by the present disclosure.

Further, the mixing ratio (weight ratio) of lecithin, polysorbate 80 and poloxamer in the second mixture is preferably 1:2 to 20. If the mixing ratio of poloxamer is less than 2, the amount of poloxamer 407 is too small to make the sol-gel phase transition difficult so that it may exist as a liquid at room temperature. If it exceeds 20, the viscosity becomes too high and thus the gel may already be a gel state at room temperature without a sol-gel phase transition.

The third mixture is preferably stirred to be sufficiently mixed. Here, the stirring method is not particularly limited, and any method capable of achieving the intended purpose of the present disclosure may be applied.

S40: the third mixture and an aqueous hyaluronic acid solution are mixed to form a fourth mixture (a complex of lecithin, poloxamer and hyaluronic acid).

The aqueous hyaluronic acid solution can delay the rapid absorption of the hydrogel in the body due to the hyaluronic acid network having a high molecular weight. As described below, when a predetermined drug (for example, a water-soluble or non-aqueous drug) is administered together with the aqueous hyaluronic acid solution, the persistence of efficacy may be extended.

With nanoparticles, hyaluronic acid, which is composed of a temperature-sensitive hydrogel can further enhance the physical properties of the hydrogel due to its structural characteristics having a longer chain structure than nanoparticles, along with the reinforcement of physical properties through the phase transition to gel in the body as described above.

Further, the nanoparticles constituting the temperature phase transition nanoparticle/hydrogel complex has both positive and negative polarities, and hyaluronic acid has negative polarity so that the polar interaction between nanoparticles and hyaluronic acid, nanoparticles and drugs, and drugs and hyaluronic acid, induces the sustained release of the drug. Thus, it enables sustained release along with local drug release along with temperature sensitivity. This is a reason that existing hydrogel has a relatively rapid drug release characteristic, but the quick drug release can be overcome by utilizing the polar interaction through the nanoparticles. Thus, it is expected that a nanoparticle/hydrogel complex is an advance over the existing hydrogel.

Here, the concentration of the aqueous hyaluronic acid solution is not particularly limited, and as described below, the ratio between the content of the hyaluronic acid and the content of the poloxamer is essential. However, the concentration of the hyaluronic acid aqueous solution may be 5% to 95%, 10% to 90%, 15% to 85%, 20% to 80%, and 25% to 75% by weight, as an example, and as described above, the concentration of the aqueous solution can be controlled and used so as to match the content as intended by the present disclosure.

Further, the mixing ratio (weight ratio) of hyaluronic acid and poloxamer is preferably 1:20 to 200. If the mixing ratio of poloxamer is more than 200, the hyaluronic acid is mixed too little, so that even if it gels in the sol-gel phase transition, there is little hyaluronic acid, which is a huge polymer, and thus the mechanical properties are incomplete, and the drug burst may occur. On the other hand, when the mixing ratio of poloxamer is less than 20, the content of hyaluronic acid becomes relatively too high, and the viscosity becomes too high even at room temperature, so it may be difficult to inject the final product into the human body using a syringe.

It is preferable that the fourth mixture is sufficiently stirred. Here, the stirring method is not particularly limited, and any method capable of achieving the intended purpose of the present disclosure may be applied.

S50: the fourth mixture is freeze-dried to form a nanoparticle/hydrogel complex (a complex having both nanoparticles and hydrogels).

The method of freeze-drying is not particularly limited, and a known method of freeze-drying may be applied, and preferably, any method may be applied unless the method deviates from the scope of the intended invention.

The nanoparticle/hydrogel complex may be provided by the method described above.

Another embodiment of the present disclosure provides a method for preparing a drug-unloaded injection solution comprising mixing the nanoparticle/hydrogel complex prepared by the above-described method with the injection solution.

The injection solution contains predetermined drugs. The nanoparticle/hydrogel complex does not have any particular effect in the body by itself, but it is injected locally into the body in a state that is simply mixed with the drug dissolved in the injection solution, thereby changing to a gel form at body temperature, so that the drug contained in the nanoparticle/hydrogel mixture complex can be slowly released.

The prepared nanoparticle/hydrogel complex does not have a therapeutic effect in the body, but may have a therapeutic effect through simple mixing with an injection solution that has been used in clinical practice.

Drugs are substances whose main purpose exhibits physiological activity, and for example, may include one or more selected from anesthetics, analgesics, antiangiogenic agents, vasoactive agents, anticoagulants, cytotoxic agents, neurotransmitters, anticancer agents, antibiotics, antiviral agents, appetite reducing agents, antiarthritic agents, anti-asthma agents, anticonvulsants, antidepressants, antihistamines, anti-inflammatory agents, antiemetics, anti-migraine drugs, anti-tumor drugs, anti-itch drugs, anti-psychotics, antipyretics, antispasmodics, cardiovascular drugs (including calcium channel blockers, beta blockers, beta agonists, or antiarrhythmics), antihypertensive agents, chemotherapeutic agents, diuretics, vasodilators, central nervous system stimulants, cough and cold agents, decongestants, diagnostic agents, hormones, bone formation stimulators and bone resorption inhibitors, immunomodulators, immunosuppressants, muscle relaxants, psychotropic drugs, psychostimulants, sedatives, tranquilizers, proteins, peptides (including those formed by naturally occurring, chemical synthesis or recombination), nucleic acid molecules (including polymer forms of two or more nucleic acids, ribonucleotides or deoxyribonucleotides including double and single-stranded molecules and supercoiled or condensed molecules, gene constructs, expression vectors, plasmids, antisense molecules, etc.), antibodies, lipids, cells, tissues, vaccines, genes, and polysaccharides.

In other words, the frozen powder according to the present disclosure is an excellent drug carrier and loads the various aforementioned drugs, thereby sufficiently prolonging the release time of the drug due to the interaction of nanoparticles, hydrogels, and drugs when injected into the body.

Therefore, conventionally, when a drug is specified, a drug carrier suitable for the drug should be manufactured, but the drug carrier according to the present disclosure excludes the drug, and after manufacturing only the drug carrier, a drug suitable for the user's intention is selected as needed. Thus, it can be easily mixed and used. Further, as described above, when it is injected into the body, the release time of the drug can be sufficiently extended. In addition, lecithin, a biocompatible material, can be used to help regenerate cartilage.

Further, another embodiment of the present disclosure provides a temperature phase transition nanoparticle/hydrogel complex.

The temperature phase transition nanoparticles/hydrogel complex comprises an irregular mesh-shaped hyaluronic acid network; and nanoparticles including lecithin, polysorbate 80, and poloxamer 407 located between the network, in which the mixing ratio (weight ratio) of the hyaluronic acid and poloxamer 407 is 1:20 to 200.

As a specific example, lecithin and polysorbate 80 may be included in a weight ratio of 1:0.25, lecithin and poloxamer 407 may be included at 1:10, and poloxamer 407 and hyaluronic acid may be included at 1:25.

Further, lecithin:polysorbate 80:poloxamer 407:hyaluronic acid may be included in a weight ratio of 1:0.1 to 2:2 to 20:0.01 to 1. According to this composition range, it is possible to provide a sustained-release drug carrier having an appropriate compressive strength as intended in the present disclosure.

In the case of the same components as those described in the method of preparing the nanoparticle/hydrogel complex described above, the description thereof is excluded.

The complex consists of nanoparticles and a network of hyaluronic acid. It is obvious that additional components may be included thereto.

The nanoparticles are lipid-based nanoparticles. The nanoparticles interact with various drugs to be mixed later and can be bound to drugs such that the drugs are introduced into the body and slowly released. In the case of a hydrogel consisting only of poloxamer 407 or poloxamer 407/hyaluronic acid, when it carries a drug by mixing with a drug, it cannot hold the drug for a certain period of time through the interaction of the polymer material and the drug, so that the duration of absorption and efficacy of the drug is determined depending on the absorption rate of the polymer gel. Therefore, the present disclosure stabilizes the lipid-based nanoparticles of natural ingredients capable of carrying hydrophilic and hydrophobic drugs, and mixes them with a hydrogel having a reversible sol-gel transition behavior in response to temperature, thereby producing a nanoparticle/hydrogel complex for carrying drugs, which can extend the efficacy time by the continuous release of the drug through a complex effect, which is not a system that depends only on the absorption rate of the gel through the interaction between the effective drug to be delivered and the stabilized lipid-based nanoparticles. Further, lecithin contained in nanoparticles is an amphoteric substance having both anionic and cationic properties and can be applied not only to nonionic salt-type drugs but also to various ionic drugs. Thus, its strong interaction with lipid-based nanoparticles allows longer sustained release when administered into the body.

The hyaluronic acid network is composed of hyaluronic acid. The substance that determines temperature sensitivity is poloxamer 407. However, if it is composed of only poloxamer 407 or nanoparticles/poloxamer 407, it disappears by absorption in the body within 1 to 2 hours after administration into the body, so the efficacy of the drug loaded together with it cannot be sustained. However, when sodium hyaluronate is included together, hyaluronic acid exists in the form of a high molecular weight polymer network, so that it is slowly absorbed in the body. Therefore, the temperature-sensitive hydrogel containing sodium hyaluronate is also slowly absorbed so that the duration of the drug effect may be extended by prolonging the absorption time of the drugs contained together with it.

Further, the nanoparticle/hydrogel complex is in a sol state at room temperature, or a gel state at 31° C. or higher. Preferably, the gel state is maintained at 31° C. to 36° C.

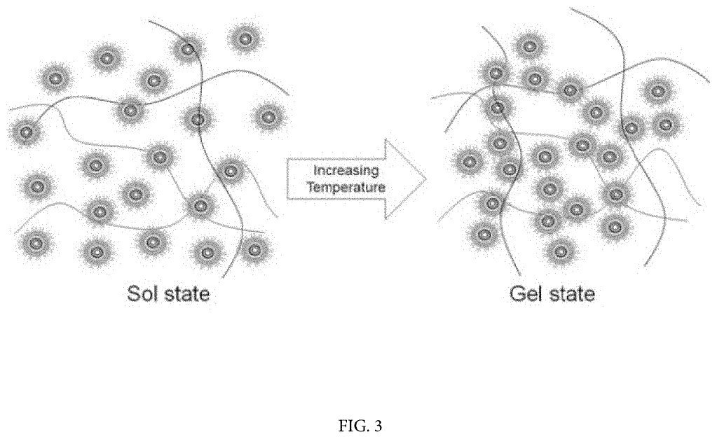

FIG. 3 is a schematic view for explaining a sol-gel phase transition phenomenon according to an increase in temperature of a nanoparticle/hydrogel complex for sustained-release drug delivery according to an embodiment of the present disclosure. As shown in FIG. 3, when mixed with an aqueous solution containing a drug and a hydrogel containing nanoparticles, the nanoparticles are uniformly dispersed in the aqueous solution at room temperature, but a solid gel is formed through the interaction between the nanoparticles at a temperature similar to the body temperature as the temperature increases.

Another embodiment of the present disclosure provides a sustained-release drug carrier solution carrying a drug including the above-described nanoparticle/hydrogel complex, the predetermined drug dispersed in the complex and the injection solution.

Hereinafter, the present disclosure will be described in more detail through experimental examples.

### Experimental Example 1. Confirmation of Morphology of Nanoparticle/Hydrogel Complex

First, 2 ml of a 10% aqueous lecithin solution and 50 mg of polysorbate 80 were mixed to form about 2 ml of the first mixture. Then, the first mixture was irradiated with ultrasonic irradiation (probe type) to form about 2 ml of the second mixture containing nanoparticles having a size of about 150 nm. Then, the second mixture and 20 ml of a 10% aqueous poloxamer solution were mixed to form about 22 ml of the third mixture. Then, about 22 ml of the third mixture and 8.350 ml of an aqueous hyaluronic acid solution having a concentration of 0.6% were mixed to form the fourth mixture. Finally, the fourth mixture was freeze-dried to form a nanoparticle/hydrogel complex.

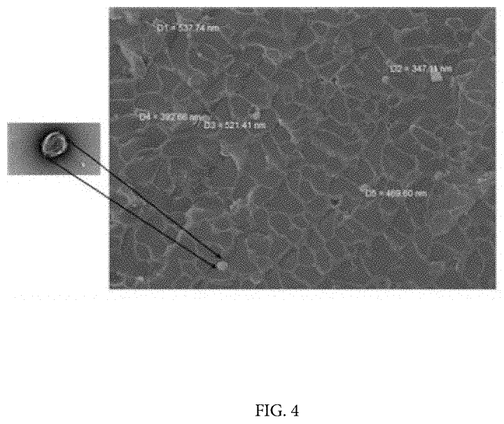

In order to confirm the morphology of the nanoparticle/hydrogel complex of the present disclosure, the frozen powder was observed with a scanning electron microscope. It was measured using cryo-SEM, and the image is shown in FIG. 4.

As shown in FIG. 4, it was confirmed that nanoparticles were formed in the structure of the nanoparticle/hydrogel complex.

### Experimental Example 2. Mechanical Properties of Nanoparticle/Hydrogel Complex

In order to confirm the mechanical properties (compressive strength) of the nanoparticle/hydrogel complex of the present disclosure, the following experiment was performed.

A mixture of the frozen powder of the nanoparticle/hydrogel complex prepared in Experimental Example 1 and saline solution (Example 1) was prepared, and a mixture of the hydrogel without nanoparticles and saline solution (Comparative Example 1) was used as a control.

The Stress-Strain characteristics of Example 1 and Comparative Example 1 were measured by a compression test (compression speed: 1 mm/min) using a universal testing machine (UTM: Universal Testing Machine, AG-X, Shimadzu, Japan). Gel-diameter was 6.7 mm, the thickness was 8 mm, and the load cell was 500N.

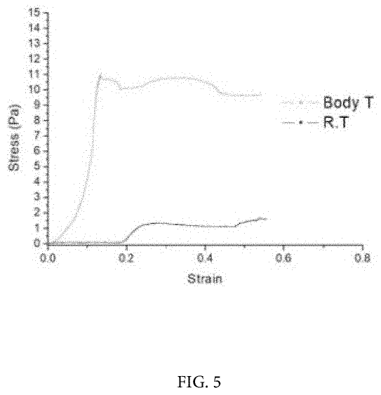

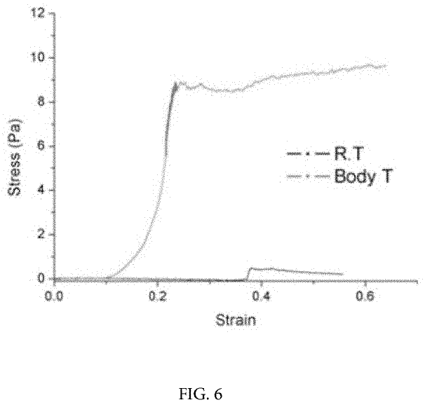

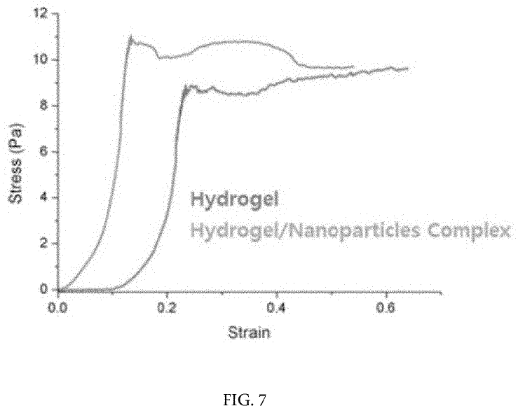

Example 1 was measured at room temperature and body temperature, and a graph of the results is shown in FIG. 5. In addition, Comparative Example 1 was measured at room temperature and body temperature, and a graph of the results is shown in FIG. 6. A comparison graph is shown in FIG. 7 to compare the results measured at body temperature for Example 1 and Comparative Example 1.

As shown in FIGS. 5 to 7, mechanical properties showing ten times or more viscous properties were confirmed at body temperature compared to room temperature. The evidence confirms that Example 1 was in a sol state at room temperature, but was formed in a gel state when introduced into the body and that mechanical properties of Example 1 were enhanced compared to Comparative Example 1.

### Experimental Example 3. Confirmation of Drug Release Behavior of Nanoparticle/Hydrogel Complex

In order to confirm the drug release behavior of the nanoparticle/hydrogel complex of the present disclosure, the following experiment was performed.

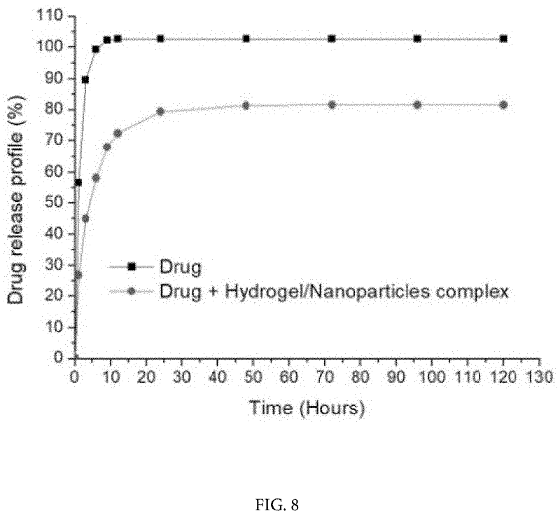

Comparative Example 2 is a case in which only the drug (aqueous solution of ropivacaine hydrochloride) was injected (Comparative Example 2), and Example 2 is a case in which 10 ml of ropivacaine hydrochloride was mixed with the frozen powder prepared in Experimental Example 1. For Comparative Example 2 and Example 2, tests were conducted using a semipermeable membrane having a size of 12,000 to 14,000 kDal to confirm the drug release behavior according to time. Each sample was collected at 1, 3, 5, 7, 9, 12, 24, 48, 72, 96, 120 hours, and the amount of drug release was quantified by high performance liquid chromatography (HPLC). The following conditions were used, and the resulting graph is shown in FIG. 8.

Mobile phase: Acetonitrile (ACN): pH 8.0 buffer=6:4

Injection volume: 20 ul

Column: ODS HYPERSIL (150 mm×4 mm)

Flow rate: 1.2 ml/min

Wave length: UV 240 nm

As shown in FIG. 8, it was confirmed that Example 2 showed a lower release rate compared to the case where only the drug of Comparative Example 2 was injected. This evidence demonstrates that Example 2 of the present disclosure shows sustained-type drug release.

### Experimental Example 4. Confirmation of Drug Release Behavior of Nanoparticle/Hydrogel Complex According to the Content of Lecithin

Further, in order to confirm the drug release behavior of the nanoparticle/hydrogel complex of the present disclosure, the following experiment was performed.

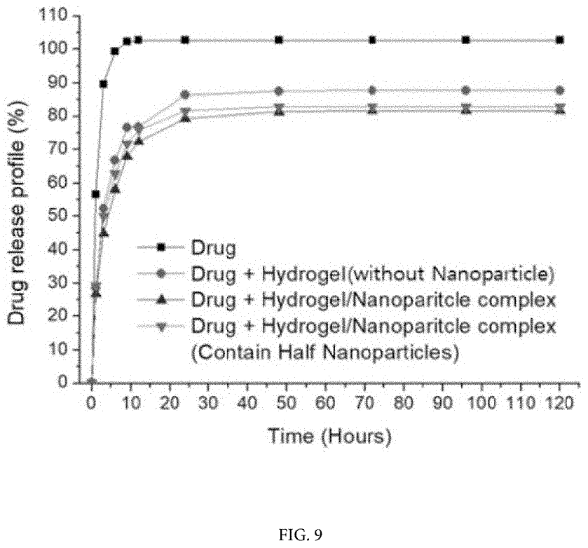

Comparative Example 3 is a case in which only a drug is injected, Comparative Example 4 is a case in which a drug is mixed and injected with a hydrogel, Example 3 is a case in which a drug is mixed and injected with nanoparticle/hydrogel complex, and Example 4 is a case in which a drug is mixed and injected with nanoparticle/hydrogel complex in a half content compared to the nanoparticle content of Example 3. For Comparative Examples 3 and 4 and Examples 3 and 4, tests were conducted using a semipermeable membrane having a size of 12,000 to 14,000 kDal to confirm the drug release behavior according to time. Each sample was collected at 1, 3, 5, 7, 9, 12, 24, 48, 72, 96, 120 hours, and the amount of drug release was quantified by high-performance liquid chromatography (HPLC). The following conditions were used, and the resulting graph is shown in FIG. 9.

As shown in FIG. 9, the drug release behavior according to the change in the content of lecithin confirms that when the lecithin content in the temperature phase transition nanoparticles/hydrogels for sustained-release drug delivery was increased, the drug was released more slowly.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method of preparing a temperature phase transition nanoparticle/hydrogel, the method comprising the steps of:
forming a first mixture by mixing an aqueous lecithin solution and polysorbate 80;
forming a second mixture including nanoparticles by irradiating ultrasonic waves to the first mixture;
forming a third mixture by mixing the second mixture and an aqueous poloxamer solution;
forming a fourth mixture by mixing the third mixture and an aqueous hyaluronic acid solution; and
forming a nanoparticle/hydrogel complex by freeze-drying the fourth mixture.

2. The method of claim 1, wherein the mixing ratio (weight ratio) of the lecithin and polysorbate 80 is 1:0 to 2 in the step of forming the first mixture.

3. The method of claim 1, wherein the average particle diameter of the nanoparticles is 40 nm to 250 nm in the step of forming the second mixture.

4. The method of claim 1, wherein the poloxamer is a poloxamer 407.

5. The method of claim 1, wherein the mixing ratio (weight ratio) of lecithin and polysorbate 80, and poloxamer in the second mixture is 1:2 to 20 in the step of forming the third mixture.

6. The method of claim 1, wherein the aqueous hyaluronic acid solution includes hyaluronic acid.

7. A temperature phase transition nanoparticles/hydrogel complex comprising:
an irregular mesh-shaped hyaluronic acid network; and
nanoparticles including lecithin, polysorbate 80, and poloxamer 407 located between the network,
wherein the mixing ratio (weight ratio) of the hyaluronic acid and poloxamer 407 is 1:20 to 200.

8. The nanoparticles/hydrogel complex of claim 7, wherein a weight ratio of the lecithin, polysorbate 80:poloxamer 407:hyaluronic acid is 1:0.1 to 2:2 to 20:0.01 to 1.

9. The nanoparticles/hydrogel complex of claim 7, wherein the nanoparticle/hydrogel complex is a sol state at room temperature, but a gel state at 31° C. or higher.

10. The nanoparticles/hydrogel complex of claim 7, wherein the nanoparticle is a lipid-based nanoparticle.
